(19) United States
(12) Patent Application Publication
(10) Pub. No.: US 2006/0122140 A1
(43) Pub. Date: Jun. 8, 2006

(54) In Vitro and in vivo silencing of human c-myc oncogene expression by poly-DNP-RNA

(76) Inventors: Jui H. Wang, Williamsville, NY (US); Long Shen, Tonawanda, NY (US); Chongjie Zhang, Chengdu (CN); Julian L. Ambrus JR., Buffalo, NY (US)

Correspondence Address: HODGSON RUSS LLP, ONE M & T PLAZA, SUITE 2000, BUFFALO, NY, 14203-2391, US

(21) Appl. No.: 11/284,693

(22) Filed: Nov. 22, 2005

Related U.S. Application Data
(60) Provisional application No. 60/630,084, filed on Nov. 22, 2004.

(51) Int. Cl. A61K 48/00

(52) U.S. Cl. 514/44

(57) ABSTRACT

The present invention provides antisense oligoribonucleotides with sequences that recognize and bind to the c-myc gene or c-myc mRNA, where one or more sugar residues of the oligoribonucleotides are modified by the substitution of DNP at the 2′-O position. The antisense oligoribonucleotides can be used for inhibiting the growth of cells in which the c-myc gene is overexpressed by down-regulation of transcription of c-myc RNA or translation of the c-myc protein. The antisense oligoribonucleotides can also be used for diagnostic purposes to identify the overexpression of the c-myc gene. The nucleotide sequence of the DNP-oligoribonucleotides is complementary to the sequence 578 to 598 in c-myc mRNA and does not contain a G-quartet motif. This DNP-RNA can silence c-myc gene expression both in vitro and in vivo with high efficacy and sequence specificity.

This application claims priority to U.S. Provisional Application No. 60/630,084, filed on Nov. 22, 2004, the disclosure of which is incorporated herein by reference.

### FIELD OF THE INVENTION

This invention relates generally to antisense oligonucleotides and more particularly to antisense poly-DNP oligoribonucleotides.

### BACKGROUND

Ever since Bishop and his co-workers discovered the c-myc gene in the late 70s (1), voluminous literature has documented its pivotal role in the proliferation and malignant transformation of human and animal cells (2,3). Many types of human malignancy have been reported to show amplification and/or overexpression of this gene, although the frequency of these alterations varies greatly in different reports (4). For example, Burkitt's lymphoma is a frequent hematologic childhood cancer in which virtually all patients display a translocation involving chromosomes 8 and 14 that places c-myc from chromosome 8 under the transcriptional control of the immunoglobulin M heavy-chain enhancer from chromosome 14 (5).

Multiple studies using transgenic mice in which the c-myc gene was overexpressed under the control of mammary specific promoters have indicated a potential role for c-myc in the progression of breast cancer (6). The first of these studies used the MMTV (mouse mammary tumor virus) promoter to overexpress c-myc in the mammary glands of mice and resulted in spontaneous mammary adenocarcinomas in two distinct lines by 4 to 8 months of age (7). Other transgenic mice experiments also demonstrated that c-myc can induce mammary tumor formation when overexpressed in the mammary gland (8). When the c-myc gene is overexpressed under the control of immunoglobulin mu or kappa enhancer, transgenic mice frequently develop a fatal lymphoma within a few months of birth (9). More recently, several groups have generated transgenic mice that conditionally express c-myc to show that inactivation of the c-myc gene is sufficient to induce tumor regression (10,11). It is therefore possible that the targeted inactivation of c-myc could lead to an effective treatment for certain types of cancer.

Because of its important role in proliferation and malignant transformation, the c-myc proto-oncogene is a frequent target of antisense technology for the purpose of developing a lead compound for therapeutic application (12-16). However, after extensive study and clinical trials, a successful antisense compound for blocking c-myc expression is still not available. One reason for this failure is the presence of a G-quartet motif in the antisense sequence that leads to non-specific sequence-independent side effects. This G-quartet motif confers a tendency to form a quadruplex, an aggregate formed by the Hoogsteen base-pairing of four single-stranded oligonucleotides (17). Quadruplex structures have been implicated in antithrombotic activity (18) as well as in the binding to, and inhibition of, the macrophage scavenger receptor (19). Another reason for this failure may be that the antisense compound targeting the c-myc gene was developed with phosphorothioate backbone, a platform that causes non-specific cell activation by binding to cellular and extracellular proteins (20). An attempt to replace the phosphorothioate backbone with a phosphorodiamidate morpholino backbone has also been unsatisfactory due to lower efficacy and difficulty of delivery. To target the c-myc gene specifically for silencing, it is necessary to develop an antisense compound with higher efficacy and specificity.

The discovery of RNA interference (RNAi) started a new era in antisense technology (21,22). Double-stranded small interfering RNA (siRNA) were found to silence specific mRNA almost 100 times more effectively than single-stranded antisense phosphorothioate oligodeoxynucleotides (PS-ODN) (23). It has been demonstrated that only the antisense strand of siRNA is incorporated into the RNA-induced silencing complex (RISC). Single stranded antisense siRNAs transfected into HeLa cells silence an endogenous gene with comparable efficiency to double stranded siRNA(24). Native single-stranded RNA can be rapidly hydrolyzed by endogenous RNases whereas the double-stranded siRNA remains more stable due to hybridization, until it is guided into the RISC and has the sense strand removed. Delivery, however, is still a problem as successful administration of RNAi often requires the use of plasmids or viral vectors; these delivery methods carry the risk of random integration into chromosomal DNA, a serious threat to therapeutic safety. (Stevenson, M. (2004) N. Engl. J. Med., 351, 1772-1777.) Therefore if the antisense strand can be stabilized by chemical modification without interfering with its basic biochemical properties, the resulting single stranded antisense RNA could be just as potent or an even better inhibitor of gene expression than native siRNA.

One type of promising oligonucleotide derivative is poly-2′-O-(2,4-dinitrophenyl)-oligoribonucleotide (DNP-RNA) which can improve the stability and membrane permeability of antisense RNA and still maintain its high affinity with mRNA to silence the targeted gene expression. It was found that DNP-RNAs with DNP/nucleotide molar ratio of about 0.7 can spontaneously cross viral envelopes and mammalian cell membranes (25). They are also resistant to degradation by ribonucleases (26) and can hybridize with their complementary RNA with greater affinity than DNA, Mixed Backbone Oligodeoxynucleotide (MBO) or native RNA (27). Several antisense DNP-RNAs have been synthesized and found to be effective anti-cancer or antiviral agents in a sequence-specific and concentration-dependent way with no sequence-independent general toxicity in the effective concentration range (28-31).

Despite previous studies, there is an ongoing need for a successful antisense compound for blocking c-myc expression.

### SUMMARY OF THE INVENTION

The present invention provides antisense oligoribonucleotides which have complementary nucleic acid sequences that recognize and bind the c-myc gene or its mRNA resulting in the down-regulation of c-myc gene expression.

The antisense oligoribonucleotides of the present invention can be used for down-regulating the expression of the c-myc gene. In one embodiment, the antisense oligoribonucleotides can be used to inhibit the growth of cancer cells.

The antisense oligoribonucleotides of the present invention can be administered in a suitable pharmaceutical carrier to an individual to effect down-regulation of the c-myc gene. In one embodiment, the antisense oligoribonucleotide is administered to an individual in which the c-myc gene is overexpressed. Accordingly, these compositions can be used in a wide variety of conditions for inhibiting the growth of cells in which the c-myc gene is overexpressed, and accordingly for the treatment of diseases related to such overexpression. In the antisense oligoribonucleotides of the present invention, one or more sugar residues are modified by the substitution of DNP at the 2′-O position.

The antisense oligoribonucleotides of the present invention can also be used for diagnostic purposes to identify the overexpression of the c-myc gene.

In one embodiment, the oligoribonucleotide of the invention has 21-30 nucleotides comprising at least the 21 contiguous nucleotides of SEQ ID NO:1 or one-base mismatch therefrom.

In one embodiment, the base sequence of the DNP-RNA provided herein is complementary to the sequence 578 to 598 in c-myc mRNA, located at +20 downstream from the AUG starting site. It does not contain the G-quartet motif which is believed to cause non-specific general toxicity. This DNP-RNA can silence c-myc gene expression both in vitro and in vivo with high efficacy and sequence specificity.

### DESCRIPTION OF THE DRAWINGS

FIGS. 1A, 1B and IC depict graphical representations of sequence and concentration dependence of the inhibition of cell growth by poly-DNP-RNAs in MCF-7 breast cancer cells, A549 lung adenocarcinoma cells, and Colo829 melanoma cells, respectively. The cells were plated at a concentration of 2×10⁴ per well then treated with different concentrations of poly-DNP-RNAs in the presence of oligofectamine. After incubation for 8 days, cell proliferation rate was determined by using CellTiter 96® AQᵤₑₒᵤₛ One Solution Cell Proliferation Assay from Promega. Data are expressed as the percentage of growth inhibition in reference to the growth of untreated control cells. The data are presented as means±SD of four independent determinations.

FIG. 2A is a photographic representation of electrophoretic separation of RT-PCR amplification products demonstrating the in vitro gene silencing effect of poly-DNP-RNAs on the steady-state concentration of mRNAs in MCF-7 cells. The amplified cDNA bands are shown in eight lanes on the right of the molecular weight ladder in 1.5% agarose gel. Lanes 1-4 are amplified with c-myc primers, Lanes 5-8 are amplified with actin primers. Lane 1 & 5, cells without any treatment. Lane 2 & 6, cells treated with antisense poly-DNP-RNA. Lane 3 & 7, cells treated with sense poly-DNP-RNA. Lane 4 & 8, cells treated with mismatched poly-DNP-RNA.

FIG. 2B is a graphical representation of ratios of Real-time RT-PCR products from MCF-7 cells. Cells were harvested, RNA extracted, and cDNA synthesized. Real-time RT-PCR was performed as described in Example 3. Each ratio was normalized to Eukaryotic 18S rRNA Endogenous Control and converted to percentage based on the same ratio in nontreated cells. Each point represents averages from three independent experiments; bars, ±SE. CK: nontreated cells, AS 21: cells treated with antisense poly-DNP-RNA, SEN 21: cells treated with sense poly-DNP-RNA, MIS 21: cells treated with mismatched poly-DNP-RNA.

FIG. 3 is a photographic representation of a Western blot analysis of c-myc protein expression level in MCF-7 cells. Lysate from MCF-7 cells was probed with mouse anti-human c-myc monoclonal antibody (clone 9E10), c-myc was identified as a band of 62 KDa. Treatments were as follows: Lane 1, untreated control; Lane 2, treated with AS 21 antisense poly-DNP-RNA; Lane 3, treated with SEN 21 sense poly-DNP-RNA; Lane 4, treated with MIS 21 mismatched poly-DNP-RNA.

FIG. 4A is a graphical representation of the in vivo gene silencing effect of poly-DNP-RNAs on the steady-state concentration of mRNAs in c-myc transgenic mice. For all treated mice, total RNAs were isolated from blood samples collected at different time points. Day 0, blood samples collected before treatment, Day 1, 2 and 3, blood samples collected 24, 48 and 72 hours post-treatment. To produce the results shown in this figure, total RNAs from 4 transgenic mice (mouse A, B, C and D) treated with DNP modified antisense RNA inhibitor (AS-21) were assayed by real time RT-PCR as described in Example 3. The result was first normalized to Eukaryotic 18S rRNA Endogenous Control and then converted to percentage based on the same ratio of Day 0 of each mouse respectively. Each point represents averages from three independent experiments; bars, ±SE.

FIG. 4B is a photographic representation of electrophoretic separation of total RNAs from transgenic mice treated with DNP modified mismatch control (MIS-21) (mouse E) or treated with native double stranded siRNA (mouse F) were assayed by RT-PCR to evaluate the change of gene expression at mRNA level. Lanes at the upper level were amplified with c-myc primer as listed under Materials and Methods whereas lanes at the lower level were amplified with β-actin primer to serve as internal control to normalize the result. Both mouse E and mouse F showed absence of inhibition.

FIG. 5 is a graphical representation of the formation of IgG Antibody in mice after immunization with DNP-RNA and DNP-Albumin for up to 28 days. Sera were collected at different time point (Day 0, 14 and 28) from balb/c mice immunized either with PBS (CK), DNP-RNA, DNP-RNA+IFA, DNP-Albumin or DNP-Albumin+IFA, respectively. ELISA assay was performed to evaluate the production of IgG antibody against DNP group after immunization. Each point represents averages from three independent experiments; bars, ±SE.

### DESCRIPTION OF THE INVENTION

The present invention provides antisense oligoribonucleotides which have complementary nucleic acid sequences that can recognize and bind to target regions of the c-myc gene or transcribed mRNA, resulting in the down-regulation of c-myc gene expression.

The oligoribonucleotides of the present invention include sequences which are not strictly antisense i.e., these sequences may have some bases which are not complementary to the bases in the sense strand but still have enough binding affinity for c-myc mRNA to inhibit the growth of cells. Further, base modifications such as inosine in the oligoribonucleotides are contemplated to be within the scope of this invention.

As used herein, the letters “A” or “a” refer to adenine, “G” or “g” refer to guanine; “C” or “c” refer to cytosine; “T” or “t” refer to thymine; and “U” or “u” refer to uracil.

The oligoribonucleotides of the present invention are exemplified in the following 21 nucleotide sequence 5′-GGU CAU AGU UCC UGU UGG UGA-3′ (SEQ ID NO:1). One or more ribose residues in this sequence are modified at the 2′-O-position with a DNP group as shown below. All the internuclear linkages are phosphodiester bonds.

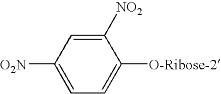

In general, the antisense oligoribonucleotide should have a sequence which is completely complementary to the targeted portion of the c-myc gene. However, absolute complementarity is not required, and a sequence with one mismatch is included within the scope of this invention. For example, it is generally known that for an oligonucleotide of about 21-25 bases, a mismatch of one base may be tolerated. One skilled in the art may readily determine the sites of other permissible mismatches from the melting point and therefore the stability of the resulting duplex. Melting points of duplexes of a given base pair composition can be determined by standard methods (see Molecular Cloning: A Laboratory Manual (J. Sambrook et al., eds)). Combination of these with cell growth inhibition data as well as protein expression and steady state mRNA data as described herein will identify the antisense oligos with permissible mismatches.

The antisense oligoribonucleotides of the present invention include sequences of 21-30 nucleotides comprising at least the 21 contiguous nucleotides of SEQ ID NO:1 or one-base mismatch therefrom. In a preferred embodiment, the antisense oligoribonucleotides include sequences of from 21 to 25 nucleotides. In a more preferred embodiment, the antisense oligoribonucleotides include sequences of from 21-23 nucleotides. In a still more preferred embodiment, the antisense oligoribonucleotides are 21 nucleotides long. One or more ribose residues of the nucleotides of the present invention are modified by the substitution of DNP at the 2′-O-position. In one embodiment, about 40-80% of the ribose residues are modified DNP. In another embodiment, about 65-75% of the ribose residues are modified by DNP. The ribose residue groups that are not modified by DNP can be modified by other groups. Such modifying groups are known in the art and include 2′-O-methyl RNA(OME), 2′-O-methoxy-ethyl RNA (MOE) and 2′-fluoropyrimidine RNA. It is preferable to have some free 2′-OH groups.

The antisense oligonucleotides are most advantageously prepared by utilizing any of the known chemical oligonucleotide synthesis methods. Therefore, oligonucleotides can be made by using commercially available, automated nucleic acid synthesizers. One such device, the Applied Biosystems 380B DNA Synthesizer, utilizes β-cyanoethyl phosphoramidite chemistry. Further, many antisense oligonucleotides are commercially available. For example, Oligo Therapeutics, Inc. has a broad line of commercially available oligonucleotides and, further, provides contract manufacturing services for the preparation of oligonucleotides. In addition, custom oligonucleotides can be made by IDT, Coralville, Iowa.

The synthesis and derivatization of single stranded RNA (ssRNA) can be carried out as follows. ssRNA is synthesized through in vitro transcription as described before (Milligan et al., 1987) with slight modification. A template containing T7 promoter can be synthesized (such as custom synthesis by commercial sources). After synthesis of the RNA, it can be derivatized by reaction with a suitable reagent such as 1-fluoro-2,4,-dinitrobenzene. The derivatized RNA is purified by the standard phenol/chloroform extraction and dialysis of the aqueous layer against water. The ratio of DNP to RNA and the actual concentration of poly DNP-RNA can be calculated from the observed absorbance at 260 and 330 nm since the oligonucleotide has absorbance only at 260 nm, whereas the DNP exhibits absorbance at both wavelengths. For larger scale synthesis, the product can be separated from the reaction mixture by column adsorption and gradient elution instead of dialysis.

For administration to individuals, the antisense oligoribonucleotides of the present invention can be incorporated into convenient pharmaceutical dosage forms such as capsules, tablets, injectable, topical or inhalable preparations. Solid or liquid pharmaceutical carriers can be employed. Solid carriers include, for example, starch, calcium, sulfate dehydrate, terra alba, sucrose, talc, gelatin, agar, pectin, acacia, magnesium stearate, and stearic acid. Liquid carriers include, for example, syrup, peanut oil, olive oil, saline and water. Liposomal, viral vector, and protein conjugate preparations can also be used as carriers. Similarly, the carrier or diluent may include any prolonged release material, such as glyceryl monostearate, or glyceryl disteararate, alone or with wax. The amount of solid carrier varies widely but preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension. When a liquid carrier is used, it will most often be a saline solution or phosphate buffered solution. For intranasal delivery, aerosolized preparations can be used.

Pharmaceutical preparations can be made following conventional techniques of a pharmaceutical chemist involving mixing, granulating and compressing, when necessary, for tablet forms, or mixing, filling, and dissolving the ingredients, as appropriate, to give the desired oral or parenteral products.

Efficacious non-toxic doses of the antisense oligoribonucleotides can be determined by clinicians having ordinary skill in the art. Typically, the dose may be selected such that it results in an extracellular concentration in the vicinity of the target cells that corresponds to what has been found to be effective as shown herein. Typically, the dose may be selected from a range of 0.1 mg/kg to about 100 mg/kg, but is preferably less than 1 mg/kg. The dose can be administered to an individual, orally, rectally, by injection, such as by such as by intramuscular, intravenous, intraarterial, intraperitoneal, or subcutaneous injection, or topically, by inhalation or continuously. It may also be delivered to the target site, such as a tumor, directly. When administered directly to the target site, a lower dose would be required.

Even in the absence of transfection agents, poly-DNP-RNAs are slowly but spontaneously transported through mammalian cell membranes (Ashun et al., 1996; Ru et al., 1999). They are also not only resistant to hydrolysis by RNases, but may actually inhibit RNases including RNase H (Rahman et al., 1996). These last two factors may also contribute to the unusually high efficacy of poly-DNP-RNA seen here.

The use of poly-DNP-RNA as a bioavailable platform for antisense RNAs has been demonstrated in the following publications: (1) Wang, A. and Wang, J. H. (1999) “Treatment of murine leukemia with poly-DNP-RNA”, Antisense & Nucleic Acid Drug Development 9, 43-51; (2) Ru, K., Schmitt, S., James, W. I. and Wang, J. H. (1999) “Antitumor effect of antisense poly-DNP-RNA in vivo”, Oncology Research 11, 505-572; (3) Ru, K., Taub, M. L. and Wang, J. H. (1998) “Antisense poly-DNP RNAs as specific breast cancer inhibitor”, Oncology Research 10, 389-397; (4) Xin, W. and Wang, J. H. (1998), “Treatment of duck hepatitis B by poly-DNP-RNA”, Antisense & Nucleic Drug Development 8, 459-468.

The present oligoribonucleotide can be administered to an individual including humans in which the c-myc gene is overexpressed. As previously discussed, this gene has been reported to be overexpressed in proliferative disorders such as cancer. In the use of the oligonucletides of this invention, both single stranded as well as double stranded poly-DNP oligoribonucleotides can be used. (Chen, et al. Drug. Discov. Today. (2005) April 15;10(8):587-93.) The oligoribonucleotides may be administered so as to effect a reduction in the growth of both tumor and non-tumor cancer cells which overexpress the c-myc gene. Examples of such cells include but are not limited to lung cancer cells, breast cancer cells, melanoma cells, lymphoma cells, leukemia cells, bladder cancer cells, colon cancer cells, gastric cancer cells, hepatica cancer cells, myeloma cells, ovarian cancer cells, prostate cancer cells and sububgual cancer cells. Further, oligoribonucleotides may be administered to mitigate non-oncological diseases believed to be cause by c-myc overexpression, such as cardiovascular restenosis. The regimen may comprise one or more doses given within a short period of time or over an extended period of time. The present antisense oligoribonucleotide may be administered alone or in combination with other therapeutic approaches such as surgical intervention, radiation, immunotherapy or chemotherapy.

Since the poly-DNP-RNA of the present invention can be used to arrest or inhibit the growth of cells which overexpress the c-myc gene, it could also be used as an anti-cancer agent with the following advantages over other chemically modified oligoribonucleotides made by solid-state synthesis:
1. The synthesis of poly-DNP-RNA by in vitro transcription followed by one-step derivatization reaction is simpler and the product has no stereochemical heterogeneity.
2. DNP-RNAs are delivered faster into mammalian cells and remain active for days inside the cells.
3. Since only low dosage of poly-DNP-RNA is required, sequence-independent non-specific toxicity should be negligible.

The antisense oligoribonucleotides of the present invention and/or complementary sequences thereof, can also be used for diagnostic purposes such as to detect the overexpression of the c-myc gene. For example, nucleic acids (mRNA or reverse transcribed DNA) can be isolated from test and control samples and hybridization reactions carried out with the oligoribonucleotides provided herein. Hybridization of nucleic acid sequences are well known to those in the art and are used routinely. Thus, conditions for hybridization can be easily determined. Any increase in hybridization in the test sample over the control sample is indicative of overexpression of the c-myc.

This invention is further described in the examples provided below which are intended to be illustrative and are not intended to be restrictive in any way.

### EXAMPLE 1

This Example describes the preparation and derivatization of the antisense RNA oligoribonucleotides of the invention. The DNP-RNA sequences in Table 1 were synthesized as previously described (31). In brief, RNA was first synthesized by in vitro transcription, using a promoter-containing DNA-template and T7 polymerase, followed by reaction with 2,4-dinitro-1-flurobenzene under controlled conditions. To characterize the DNP-RNAs, the molar DNP/nucleotide ratio (˜0.7) and the actual concentration of DNP-RNAs were calculated from the observed absorbance at 260 and 330 nm because the oligonucleotide has absorbance only at 260 nm, whereas the DNP has absorbance at both wavelengths. The purity of poly-DNP-RNAs was determined by running the samples on 16% denaturing PAGE gels. The sequence of the poly-DNP oligoribonucleotides used in this invention is presented in Table 1, wherein each bolded and italicized base represents a mismatch. The first G at the 5′-end of SEN-21 was added to facilitate in vitro transcription by T₇ polymerase

TABLE 1

| | | |
|---|---|---|
| AS 21 | (SEQ ID NO:1) | 5′- GGU CAU AGU UCC UGU UGG UGA-3′ |
| SEN 21 | (SEQ ID NO:2) | 5′- [symbol] UC ACC AAC AGG AAC UAU GAC-3′ |
| MIS 21 | (SEQ ID NO:3) | 5′- GG [symbol] CAU AG [symbol] UCC U [symbol] U UGG [symbol] G [symbol] -3′ |

### EXAMPLE 2

This Example demonstrates the specific inhibition of cancer cell growth using the oligoribonucleotide of the invention. We used three well established cancer cell lines, MCF-7 human breast cancer cells, A549 human lung adenocarcinoma cells, and Colo829 human melanoma cells purchased from ATCC (Rockville, Md.), to test the efficacy of DNP-RNAs. MCF-7 human breast cancer cells were grown in Minimum Essential Medium (MEM) a Medium supplemented with 10% fetal bovine serum (FBS) (Invitrogen, Carlsbad, Calif.) and Insulin (5 mg/ml) (Sigma, St. Louis, Mo.). A549 human lung adenocarcinoma cells were grown in F-12 Nutrient Mixture (Ham) supplemented with 10% FBS (Invitrogen). Colo829 human melanoma cells were grown in RPMI1640 medium (ATCC, Rockville, Md.). Cells were grown in a humidified atmosphere of 95% air and 5% CO₂ at 37° C.

To perform cell proliferation assays, OLIGOFECTAMINE™ reagent (GIBCO-BRL) was used to increase the delivery of poly-DNP-RNA into cells in culture. About 1×10⁴ cells were plated on 24 well plates 1 day before the treatment. Then antisense or control DNP-RNAs listed in Table 1 were added at various concentrations in the presence of OLIGOFECTAMINE (1 μl/ml). After 1 day of incubation, the medium was removed, and fresh medium was added in the absence of DNP-RNA and OLIGOFECTAMINE. Cell proliferation was then measured on day 7 by using the CellTiter 96® AQᵤₑₒᵤₛ One Solution Cell Proliferation Assay (MTS) kit form Promega (Madison, Wis.). After Pipeting 20 μl of CellTiter 96° AQueous One Solution Reagent into each well of the 24-well assay plate containing the sample in 500 μl of culture medium, the plate was incubated for 1 hour at 37° C. in a humidified, 5% CO₂ atmosphere. The absorbance at 490 nm was recorded using AD 340 Absorbance Detector from Beckman (Fullerton, Calif.).

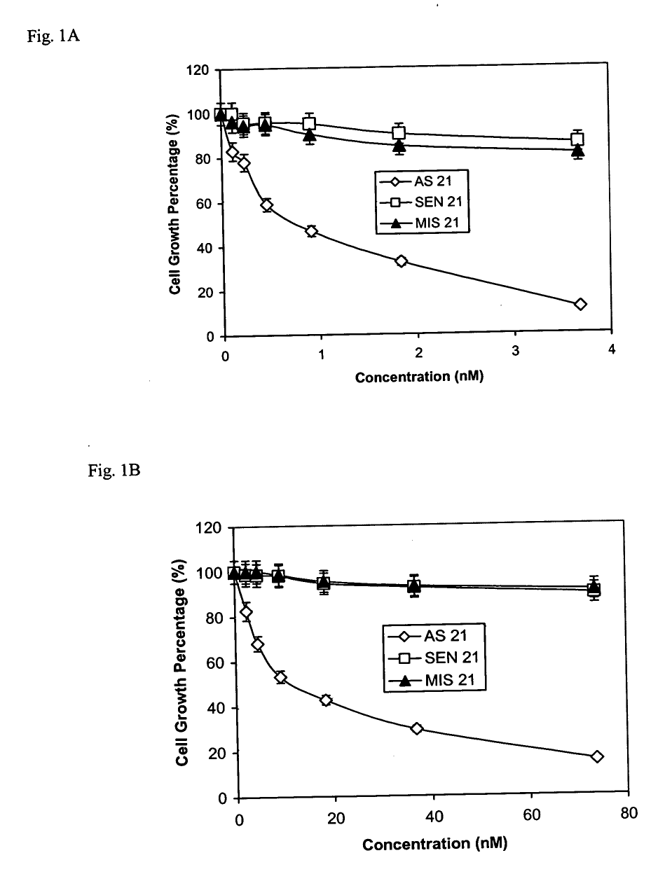

As shown in FIG. 1A, AS 21 inhibited the growth of MCF-7 human breast cancer cells in a dosage dependent manner. The estimated 50% inhibitory concentration (IC₅₀) was 0.8 nM which was more potent than those that had been reported (34). Compared to AS 21, both SEN 21 and MIS 21 controls were essentially inactive, even at the highest concentration assayed of 3.7 nM. This indicated that the cell growth inhibition effect observed for AS 21 was sequence specific.

A similar growth inhibition pattern was observed when these DNP-RNAs were added to A549 cells (FIG. 1B.). Although A549 was more tolerant to AS 21 treatment than MCF-7, AS 21 still inhibited the A549 cell growth with an estimated IC₅₀ value of 8 nM. Neither SEN 21 nor MIS 21 inhibited A549 cell growth at the highest assay concentration of 74 nM. Thus, AS 21 is more potent by at least 2 orders of magnitude than the working concentration of previously reported antisense inhibitors for the c-myc gene with different backbones, such as phosphorothioate oligodeoxynucleotide (PS-ODN)(34) and Phosphorodiamidate Morpholino oligomers (PMO)(35), which ranged from 1 μM to 100 μM.

A similar growth inhibition pattern was also observed when these DNP-RNAs were added to Colo829 cells (FIG. 1C). The estimated 50% inhibitory concentration (IC₅₀) was 0.7 nM. Compared to AS21, both the SEN21 and MIS21 were essentially inactive even at the highest concentration assayed at 3.0 nM.

### EXAMPLE 3

In this Example, the effect on c-myc mRNA expression of an oligoribonucleotide with complete complementarity to the c-myc iRNA was compared with the effect of oligoribonucleotides having bases mismatched for the c-myc mRNA sequence. Specifically, total RNA was isolated from MCF-7 cells treated with different the DNP-RNAs listed in Table 1 and assayed by both RT-PCR and Real-Time RT-PCR.

To prepare total RNA and perform RT-PCR, MCF-7 cells (2×10⁵/well) were plated in 6-well plates one day before being treated with 100 nM DNP-RNAs in the presence of OLIGOFECTAMINE. After incubation of the cells with poly-DNP-RNAs for 24 h, the cells were lysed and total RNAs were extracted using RNeasy Mini Kit (Qiagen, Valencia, Calif.). The subsequent reverse transcription was carried out in the presence 2 μl Oligo (dT)₁₂₋₁₈ (500 μg/ml) primer, reaction buffer, 0.5 mM dNTP Mix, 10 mM DTT (Invitrogen), 2 U/μl RNasin® Ribonuclease inhibitor(Promega), and 20 U/μl M-MLV reverse transcriptase (Invitrogen). The reaction was run at 39° C. for 1 h followed by incubation at 90° C. for 10 min. About 1/20 of the mixture containing the cDNA from the reverse transcription reaction was amplified with PCR in a final volume of 25 μl. The PCR reaction was performed as previously described(31) with some modification. The 30 cycles of the PCR (1 min at 94° C., 1 min at 60° C., and 1 min 30 sec at 72° C.) were preceded by 4 min of denaturation at 94° C. and followed by 5 min of elongation at 72° C. The PCR reaction mixture had a composition of IX reaction buffer [20 mM Tris-HCl (pH 8.4), 50 mM KCl], 200 μM each of dNTPs, 1.5 mM MgCl₂, 0.2 μM each of the primers and 2.5 U Platinum® Taq DNA polymerase (Invitrogen). The primer pair used for amplification of the c-myc cDNA was 5′ gac gcg ggg agg cta ttc tg (forward primer sequence SEQ ID NO4:) and 5′ agg gtg tga ccg caa cgt (reverse primer sequence SEQ ID NO:5). The primer pair used for amplification of the β-actin cDNA was 5′ gtg ggg cgc ccc agg cac ca (forward primer sequence SEQ ID NO:6 and 5′ ctc ctt aat gtc acg cac gat ttc (reverse primer sequence SEQ ID NO:7).

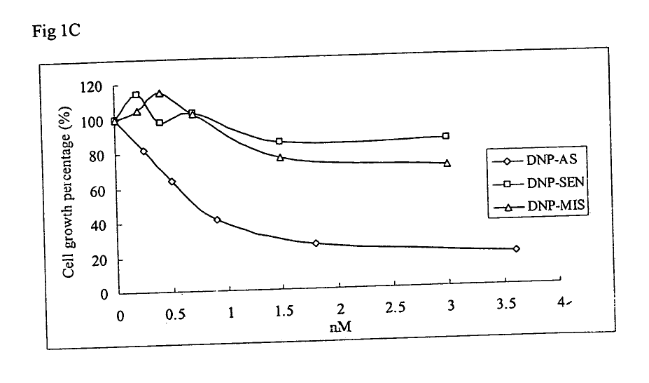

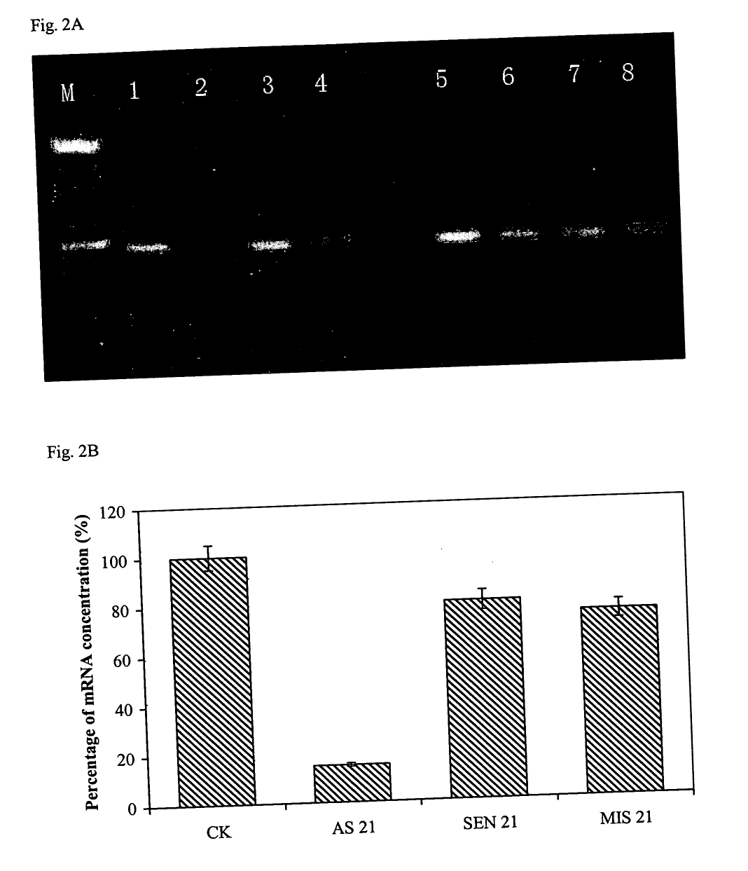

After the specific mRNAs for β-actin and c-myc were amplified by their respective primers, the relative concentrations of the corresponding cDNAs were estimated by comparing the intensities of ethidium-stained bands in the same electrophoresis gel. As shown in FIG. 2A, after treatment of MCF-7 cells with different DNP-RNAs for 24 hours, there was no significant change in the concentration of β-actin (Lane 5-8), which serves as an internal control to normalize the total RNA concentration. However, after treatment with AS 21 for 24 hours, the expression of c-myc gene in MCF-7 cells was reduced to an almost undetectable level (Lane 2). In those treated with SEN 21 (Lane 3) and MIS 21 (Lane 4) controls, the expression of c-myc gene remained the same as the untreated control (Lane 1).

To further quantify the difference between the samples treated with AS 21 and those treated with SEN 21 and MIS 21, Real-Time RT-PCR was performed by isolating total RNAs using QIAamp RNA Blood Mini Kit (Qiagen) following instructions from the manufacturer. The reagent for running Real-Time PCR was purchased from Invitrogen as SuperScript III Platinum Two-Step qRT-PCR Kits. C-myc and 18S RNA primer pairs were purchased from Applied Biosystems (Foster City, Calif.). All reagents were mixed together as instructed by the protocol provided by the manufacturer. Real-Time PCR was performed on an ABI PRISM® 7900HT Sequence Detection System (Applied Biosystems) at Roswell Park Cancer Institute.

The results are plotted as shown in FIG. 2B. If expression of c-myc gene in the untreated MCF-7 cell was set to be 100%, then after treatment with AS 21 for 24 hours, the average expression level of the c-myc gene in MCF-7 cells decreased to about 15%, whereas those treated with SEN 21 and MIS 21 remained at 80% and 75% respectively. These findings further confirmed that AS 21 can silence the expression of c-myc gene in a sequence specific manner.

### EXAMPLE 4

This Example demonstrates the effect of different DNP-RNAs on c-myc protein expression level in vitro. As a result of the breakdown of targeted mRNA after treatment with the antisense inhibitor, the subsequent protein expression should also be affected by the antisense inhibitor. A Western blot assay was performed to investigate the effect of DNP-RNAs on c-myc protein expression, and MCF-7 cells (2×10⁵/well) were plated in 6-well plates one day before being treated with 100 nM DNP-RNAs in the presence of OLIGOFECTAMINE. After incubation of the cells with poly-DNP-RNAs for 24 h, cells were washed once with PBS and lysed with 200 μl boiling lysis buffer (1% SDS, 1.0 mM sodium ortho-vanadate, 10 mM Tris pH 7.4). Protein concentration was determined by BCA Protein Assay Reagent Kit (Pierce, Rockford, Ill.), using bovine serum albumin (BSA) as a standard. Cell lysates (20 μg total proteins) were first mixed with 4×LDS loading buffer and sample reducing agent (Invitrogen). After incubation at 70° C. for 10 min, cell lysates were run on NUPAGE® 4-12% Bis-Tris Gel with MOPS running buffer and then transferred to Invitrolon® PVDF membranes (Invitrogen). Blotted membranes were developed using WesternBreeze® Novex Chromogenic Western Blot Immunodetection Kit (Invitrogen). Mouse anti-human c-myc monoclonal antibody (clone 9E10) was purchased from BD Biosciences (San Diego, Calif.).

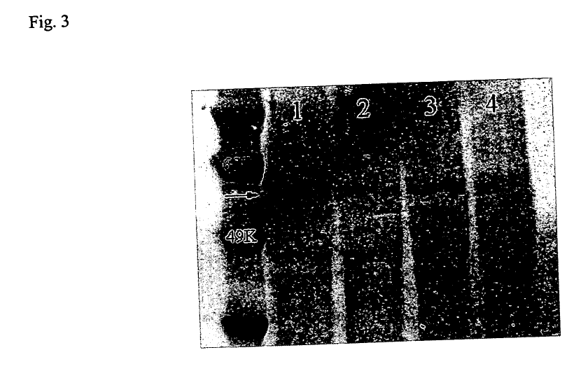

The Western blot assay results are shown in FIG. 3. The monoclonal antibodies directed against c-myc protein identified a 62-KDa band (arrow) in the whole cell extract which corresponded to the molecular mass identified in extracts of HL-60 cells, a cell line known to overexpress c-myc, which served as a positive control (data not shown). Consistent with observation from the RT-PCR assay, after 24 hour treatment with DNP-RNAs, only MCF-7 cells treated with AS 21 showed significant decrease in c-myc protein expression (Lane 2). C-myc protein expression in the MCF-7 cells treated with SEN 21 (Lane 3) and MIS 21 (Lane 4) remained the same as in the untreated MCF-7 cells (Lane 1). These Western blot data when combined with previous RT-PCR and real time PCR data strongly suggest that we have identified a sequence specific antisense inhibitor of the c-myc gene.

### EXAMPLE 5

This Example demonstrates in vivo gene silencing of c-myc by an oligoribonucleotide of the invention in C-myc transgenic mice. The mice (age 6-8 weeks) were purchased from Charles River Laboratories (Wilmington, Mass.) and maintained in the Laboratory Animal Facility at University at Buffalo. Blood samples were collected by orbital bleeding following a protocol approved by IACCU at University at Buffalo.

For animal experiments, previous works (29,30,37) demonstrated that DNP-RNAs can work efficiently without help from transfection reagents. To investigate how the DNP-RNAs work in vivo, we used the c-myc transgenic mice as a model system. These transgenic mice carry a fusion gene in which the mouse immunoglobulin enhancer has been inserted into an otherwise normal human c-myc gene. As a result the c-myc gene is overexpressed, and the transgenic mice develop pre-B cell lymphomas at age of 6-9 months (38). The presence of the transgene vector in the transgenic mice was first confirmed by PCR using the primer sequences provided by Charles River Lab. In a two-week trial period, blood samples were taken from untreated mice twice a week and the result from the RT-PCR assay indicated that the expression of the c-myc gene remained consistent for each mouse tested (data not shown). In these animal experiments, we first checked all mRNA samples with the RT-PCR assay. Significant changes in c-myc gene expression were further confirmed and quantified by the real time RT-PCR assay, as follows.

A total of 12 mice were each given one ip administration of AS 21 at a single dosage of 10 mg/kg at the beginning of the experiment. A 60-100 μl blood sample was collected from each animal every 24 hours following the injection of AS 21 for up to 72 hours. The total RNA in each sample was isolated and the expressions of c-myc gene were examined by both the RT-PCR assay (data not shown) and the real time RT-PCR assay as described above.

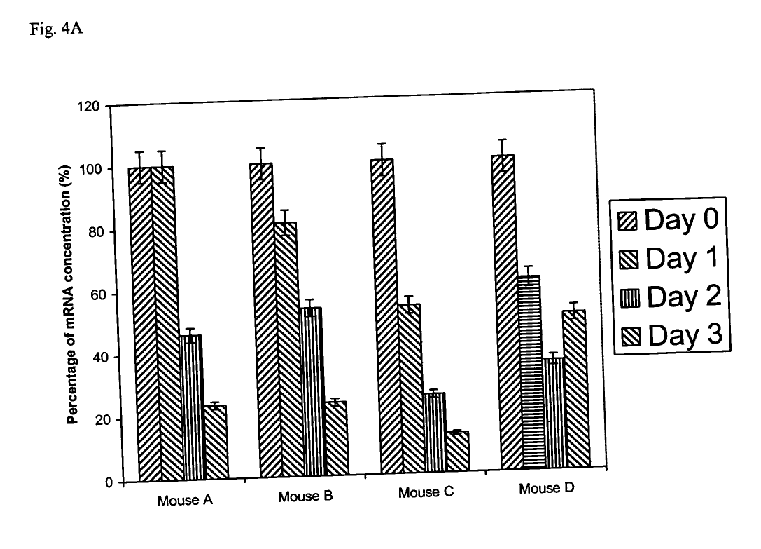

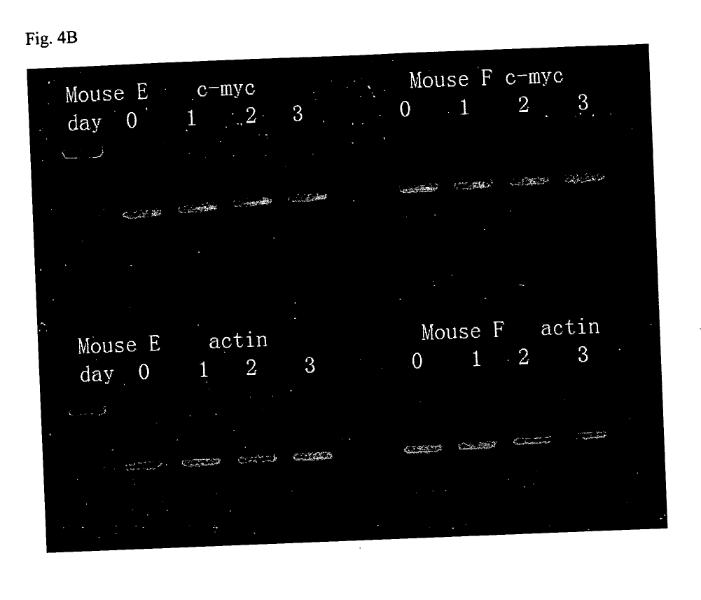

All mice demonstrated significant decreases in c-myc gene expression for up to 72 hours after the infusion of AS 21. The real time RT-PCR data from 4 mice is shown in FIG. 4 A. The expressions of c-myc for each mouse at Day 0 was considered 100%. For mouse A, there was an initial delay in the inhibitor effect. The expression of c-myc started to decrease 48 hours after treatment. At 72 hours after treatment, the expression of c-myc was lowered down to only about 23For mouse B and C, a continuous decrease of c-myc gene expression was observed for up to 72 hours. Both mice reached their lowest expression of c-myc at 72 hours, about 23% and 13% respectively. For mouse D, the decrease of c-myc gene expression reached its lowest level at 35% after 48 hours and then started to recover to 50% at 72 hours. On the average, 48 to 72 hours after treatment with AS 21, c-myc gene expression in all treated mice reached their lowest levels at 15-20%.

For comparison, a group of 6 mice were each given a 10 mg/kg dosage (ip) of MIS 21. Another group of 6 mice were each given a 20 mg/kg dosage (ip) of native siRNA which was first synthesized by in vitro transcription and then annealed together to form native double-stranded siRNA. FIG. 4B shows that administration of native siRNA did not cause significant change in the mRNA level in mouse F. As expected, treatment with the mismatched DNP-MIS 21 also had no effect on the mRNA level in mouse E. Although siRNA is already recognized as the most useful gene silencing tool for in vitro studies, the present results demonstrate that for in vivo systems DNP modified antisense ssRNA is an effective inhibitor, whereas native siRNA does not work at all. Therefore, this Example demonstrates that, in all transgenic mice treated with the AS 21 DNP antisense RNA inhibitor, expression of the c-myc gene reached its lowest level 48-72 hours post treatment (FIG. 4A.), while there was no significant change in c-myc gene expression in the transgenic mice treated with a DNP modified mismatched RNA control (FIG. 4B, mouse E). These results confirm the in vivo activity of the AS 21 oligoribonucleotide. Further, both c-myc mRNA and protein have short turnover times; the half-life of c-myc mRNA is about 0.5-1 hour (41), while the half-life of c-myc protein is 20-50 minutes (42), but one injection of DNP antisense RNA inhibitor is able to maintain gene silencing for at least 72 hours.

Thus, when transgenic mice were treated with native double stranded siRNA without the help of a transfection reagent, there was no significant change in c-myc gene expression (FIG. 4B, mouse F). Therefore, because DNP derivatization not only improves the stability of RNA (26,29) but also increases its membrane permeability (25,37), DNP antisense RNA can silence gene expression in vivo whereas native siRNA fails to show any activity.

### EXAMPLE 6

This Example demonstrates that anti-DNP antibodies are not produced in animals treated with the oligoribonucleotides of the invention.

When conjugated to protein or peptide, the dinitrophenyl (DNP) group is a good hapten that stimulates the host immune system to produce specific antibody. This raises the concern whether the DNP group in the DNP-RNA will also function as a hapten to induce host immune system reaction when DNP-RNAs are administered to the animal. To address this question, a total of 20 Balb/c mice (age 6-8 weeks) and separated into 5 groups. One group of mice was set aside as untreated controls; the other four groups were immunized with AS 21 only, AS 21 plus Incomplete Freund's Adjuvant (IFA, a drug used in vaccine therapy to stimulate the immune system), DNP-Albumin and DNP-Albumin plus IFA respectively. On day 14, after sera were collected, the mice were immunized again to boost the production of antibodies. On day 28, sera were again collected. An ELISA assay was then performed to evaluate the production of IgG antibodies following the immunization. The ELISA for anti-DNP antibodies production were performed in 96 well plates coated with DNP-KLH (keyhole limpet hemocyanin) (Biosearch Tech, Novato, Calif.) as previously described (32). Sera from the treated mice and control mice were incubated with plates, washed 3 times with PBS+0.1% Triton; then alkaline phosphatase conjugated anti-murine IgG was added as indicated. The plates were again washed with PBS+0.1% Triton and PNPP substrate (Pierce). The plates were then assayed by spectrophotometric reading at 405 nm.

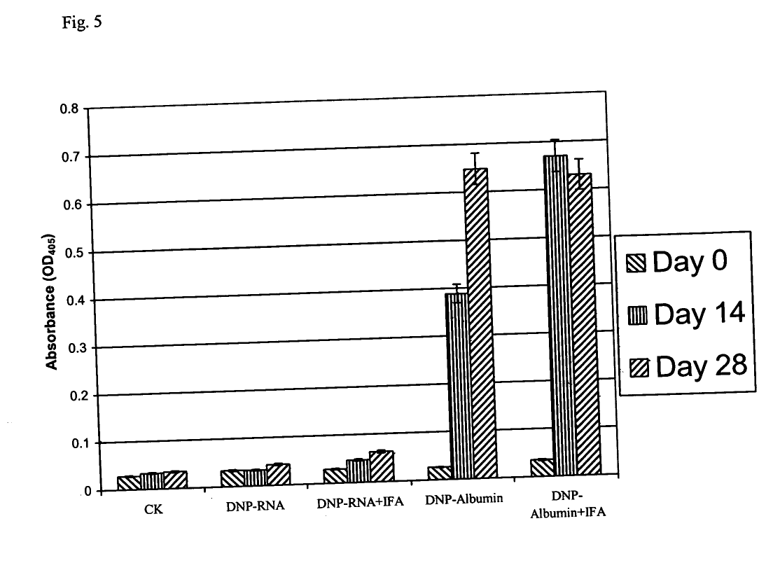

As demonstrated in FIG. 5, the untreated control group (CK) set the background level of antibody production under normal conditions. The positive control, DNP-albumin, by itself was enough to stimulate the production of DNP-specific IgG antibodies within 14 days and reached highest production at Day 28. With the help of IFA, DNP-albumin was able to enhance antibody production within 14 days and to maintain this capability for up to 28 days.

In contrast, DNP-RNA by itself did not stimulate the production of DNP specific IgG antibodies. Even in the presence of IFA, there was no significant antibody production to DNP. This indicates that DNP-RNA does not activate humoral immune responses and hence can be safely administered to live animals.

### EXAMPLE 7

This embodiment describes the use of the antisense oligoribonucleotides of the present invention to arrest or reduce the growth of malignant cells in vivo.

A human cancer xenograft model was established by standard methods (see Wang et al., 1999). Briefly, 9 SCID mice were commercially obtained (Harlan Biotech Center, Indianapolis, Ind.). Colo829 human melanoma cells purchased from ATCC (Rockville, Md.) were cultured and harvested in a medium of RPMI1640 (ATCC) plus 10% Fetal Bovine Serum (Invitrogen, Carlsbad, Calif.) and injected on Day 1 into the mice. The cells were monitored by general clinical observation, determination of body weight and tumor size. Tumor growth was recorded and tumor size calculated from the two perpendicular diameters of the implant. For determination of the effect of poly-DNP-RNAs, the oligoribonucleotides were dissolved in physiological saline (PBS) and administered to two groups of 3 mice each. Mice in Group A were treated once on Day 2 with a single intratumoral injection of 300 μg of poly-DNP-RNA. Mice in Group B were treated once every other day starting on Day 8 with 300 μg treatments of poly-DNP-RNA for seven treatments. Mice in Group C were left untreated. All 9 mice were sacrificed after 7 weeks. Tumor growth of the xenografts was studied in the treated mice and compared to growth in the untreated controls.

As shown in Table 2, mice in Groups A and B were found upon autopsy to have reduced tumor sizes as compared to the untreated mice in Group C.

TABLE 2

| | | Average | |
|---|---|---|---|
| | | tumor size | % |
| Group | Treatment Regimen | after 7 weeks | difference |
| A | Treated by intratumoral injection | 260 mm³ | −55% |
| | one time on Day 2 | | |
| B | Treated by i.p. seven times, once | 520 mm³ | −10% |
| | every other day starting on Day 8 | | |
| C | Untreated | 581 mm³ | — |

Thus, administration of the antisense oligoribonucleotides of the present invention to a mammal can reduce the growth of malignant cells in vivo.

While specific embodiments have been presented herein, routine modifications will be apparent to those skilled in the art and are intended to be within the scope of the invention.

### REFERENCES

1. Bishop, J. M. (1982) R Advances in Cancer Research, 37, 1-32.
2. Bouchard, et al. (1998) Trends in Cell Biology, 8, 202-206.
3. Dang, et al. (1999) Experimental Cell Research, 253, 63-77.
4. Nesbit, et al. (1999) Oncogene, 18, 3004-3016.
5. Magrath, I. (1992) Cancer Res, 52, 5529s-5540s.
6. Liao, et al. (2000) Endocrine-Related Cancer, 7, 143-164.
7. Stewart, et al. (1984) Cell, 38, 627-637.
8. Schoenenberger, et al. (1988) Embo J, 7, 169-175.
9. Adams, et al. (1985) Nature, 318, 533-538.
10. Felsher, et al. (1999) Molecular Cell, 4, 199-207.
11. Jain, et al. (2002) Science, 297, 102-104.
12. Heikkila, et al. (1987) A Nature, 328, 445-449.
13. Wickstrom, et al. (1988) HProc Natl Acad Sci USA, 85, 1028-1032.
14. McManaway, et al. (1990) Lancet, 335, 808-811.
15. Skorski, et al. (1997) Proc Natl Acad Sci USA, 94, 3966-3971.
16. Smith, et al. (1998) J Natl Cancer Inst, 90, 1146-1154.
17. Cheng, et al. (1997) Gene, 197, 253-260.
18. Macaya, et al. (1995) Biochemistry, 34, 4478-4492.
19. Suzuki, et al. (1999) European Journal of Biochemistry, 260, 855-860.
20. Branch, A. D. (1998) Trends in Biochemical Sciences, 23, 45-50.
21. Fire, et al. (1998) Nature, 391, 806-811.
22. Elbashir, et al. (2001) Nature, 411, 494-498.
23. Miyagishi, et al. (2003) Antisense Nucleic Acid Drug Dev, 13, 1-7.
24. Martinez, et al. (2002) Cell, 110, 563-574.
25. Ashun, et al. (1996) Antimicrob Agents Chemother, 40, 2311-2317.
26. Rahman, et al. (1996) Analytical Chem, 68, 134-138.
27. Chen, et al. (2004) Oligonucleotides, 14, 90-99.
28. Ru, et al. (1998) Oncol Res, 10, 389-397.
29. Xin, et al. (1998) Antisense Nucleic Acid Drug Dev, 8, 459-468.
30. Wang, et al. (1999) Antisense Nucleic Acid Drug Dev, 9, 43-51.
31. Shen, et al. (2003) Antisense Nucleic Acid Drug Dev, 13, 67-74.
32. Ambrus, et al. (1990) J Immunol, 145, 3949-3955.
33. Biro, et al. (1993) Proc Natl Acad Sci USA, 90, 654-658.
34. Watson, et al. (1991) Cancer Res, 51, 3996-4000.
35. Hudziak, et al. (2000) Antisense Nucleic Acid Drug Dev, 10, 163-176.
36. Roush, W. (1997) Science, 276, 1192-1193.
37. Ru, et al. Oncol Res, 11, 505-512.
38. Schmidt, et al. Proc Natl Acad Sci USA, 85, 6047-6051.
39. Woolf, et al. (1992) Proc Natl Acad Sci USA, 89, 7305-7309.
40. Sijen, et al. (2001) Cell, 107, 465-476.
41. Dani, et al. (1984) Proc Natl Acad Sci USA, 81, 7046-7050.
42. Hann, et al. (1984) Mol Cell Biol, 4, 2486-2497.

We claim:

1. A composition comprising:
a) an oligoribonucleotide of from 21-30 nucleotides comprising a contiguous sequence of SEQ ID NO:1 or a sequence which has a one base mismatch with SEQ ID NO:1;
wherein the ribose residue of at least one nucleotide is protected at the 2′-O-position by 2,4-dinitrophenyl (DNP) and wherein the oligoribonucleotide is capable of down-regulating the expression of the c-myc gene.

2. The composition of claim 1, wherein the oligoribonucleotide has from 21 to 25 nucleotides.

3. The composition of claim 2, wherein the oligoribonucleotide has from 21-23 nucleotides.

4. The composition of claim 3, wherein the oligoribonucleotide has the sequence of SEQ ID NO:1.

5. The composition of claim 1, wherein the DNP to nucleotide molar ratio is between 0.4 to 0.8

6. The composition of claim 5, wherein the DNP to nucleotide molar ratio is between 0.65 to 0.75.

7. The composition of claim 1, further comprising a complementary strand to the oligoribonucleotide.

8. The composition of claim 1, further comprising a pharmaceutically acceptable carrier.

9. The composition of claim 8, further comprising a chemotherapeutic agent.

10. The composition of claim 8, wherein the oligoribonucleotide has the sequence of SEQ ID NO:1.

11. A method of down regulating the expression of the c-myc gene in a cell comprising providing to the cell the composition of claim 1 in an amount effective to down-regulate the expression of the c-myc gene.

12. The method of claim 11, wherein the sequence of the oligoribonucleotide is SEQ ID NO:1.

13. The method of claim 11, wherein the cell is a lung cancer cell, a breast cancer cell or a melanoma cell.

14. A method of reducing the growth of cancer cells in an individual comprising administering to the individual a growth inhibiting regimen of the composition of claim 8.

15. The method of claim 14, wherein the oligoribonucleotide in the composition has the sequence of SEQ ID NO:1.

16. The method of claim 14, wherein the administration of the composition is combined with a treatment selected from the group consisting of surgery, radiation, chemotherapy and immunotherapy.

17. The method of claim 14, wherein the composition is administered via a route selected from the group consisting of intratumoral, intravenous, intraperitoneal, intramuscular, intranasal, oral, topical, inhaled and rectal.

18. The method of claim 14, wherein the composition is administered in combination with a chemotherapeutic agent.

19. The method of claim 14, wherein the cancer cells are lung cancer cells, breast cancer cells or melanoma cells.

20. The method of claim 14, wherein the composition further comprises a complementary strand to the oligoribonucleotide.


SEQUENCE LISTING (7 sequences)

SEQ ID NO: 1 - DNA, 21 bp, Artificial Sequence
  poly DNP c-myc antisense oligomer
ggucauaguuccuguugguga

SEQ ID NO: 2 - DNA, 21 bp, artificial sequence
  poly DNP c-myc sense oligomer
gucaccaacaggaacuaugac

SEQ ID NO: 3 - DNA, 21 bp, artificial sequence
  poly DNP c-myc missense oligomer
gggcauagcuccuauuggagc

SEQ ID NO: 4 - DNA, 20 bp, artificial sequence
  c-myc cDNA forward primer
gacgcggggaggctattctg

SEQ ID NO: 5 - DNA, 18 bp, artificial sequence
  c-myc cDNA reverse primer
agggtgtgaccgcaacgt

SEQ ID NO: 6 - DNA, 20 bp, artificial sequence
  beta actin cDNA forward primer
gtggggcgccccaggcacca

SEQ ID NO: 7 - DNA, 24 bp, artificial sequence
  beta actin cDNA actin forward primer
ctccttaatgtcacgcacgatttc